(19) United States
(12) Patent Application Publication
(10) Pub. No.: US 2014/0114103 A1
(43) Pub. Date: Apr. 24, 2014

(54) PROCESS FOR PREPARING CYCLOHEXANE COMPRISING A PRIOR REMOVAL OF DIMETHYLPENTANES

(71) Applicant: BASF SE, Ludwigshafen (DE)

(72) Inventors: Markus Schmitt, Heidelberg (DE); Katharina Spuhl, Forest (BE); Jochen Bürkle, Mannheim (DE); Stefan Bitterlich, Dirmstein (DE); Steffen Tschirschwitz, Mannheim (DE); Michael Hübner, Lampertheim (DE); Pawel Czajka, Mannheim (DE)

(73) Assignee: BASF SE, Ludwigshafen (DE)

(21) Appl. No.: 14/053,659

(22) Filed: Oct. 15, 2013

Related U.S. Application Data
(60) Provisional application No. 61/715,308, filed on Oct. 18, 2012.

(51) Int. Cl. C07C 5/31; C07C 5/10

(52) U.S. Cl. CPC C07C 5/31; C07C 5/10
USPC 585/264; 585/266

(57) ABSTRACT

The present invention relates to a process for preparing cyclohexane from benzene and/or methylcyclopentane (MCP) by hydrogenation or isomerization. Prior to the cyclohexane preparation, the dimethylpentanes (DMP) are removed in a distillation apparatus (D1) from a hydrocarbon mixture (HM1) comprising not only benzene and/or MCP but also DMP. If cyclohexane is already present in the hydrocarbon mixture (HM1), this cyclohexane is first removed together with DMP from benzene and/or MCP. This cyclohexane already present can be separated again from DMP in a downstream distillation step and recycled into the process for cyclohexane preparation.

This patent application claims the benefit of pending U.S. provisional patent application Ser. No. 61/715,308 filed on Oct. 18, 2012, incorporated in its entirety herein by reference.

The present invention relates to a process for preparing cyclohexane from benzene and/or methylcyclopentane (MCP) by hydrogenation or isomerization. Prior to the cyclohexane preparation, the dimethylpentanes (DMP) are removed in a distillation apparatus (D1) from a hydrocarbon mixture (HM1) comprising not only benzene and/or MCP but also DMP. If cyclohexane is already present in the hydrocarbon mixture (HM1), this cyclohexane is first removed together with DMP from benzene and/or MCP. This cyclohexane already present can be separated again from DMP in a downstream distillation step and recycled into the process for cyclohexane preparation.

Cyclohexane is an important product of value in the chemical industry, which can be prepared, for example, by hydrogenation of benzene or by isomerization of methylcyclopentane (MCP). However, the reactants usable for cyclohexane preparation (benzene or MCP) are in practice generally not in the form of pure substances, but a constituent of hydrocarbon mixtures. The specific composition of the hydrocarbon mixtures can vary greatly. Frequently, benzene and/or MCP are present in hydrocarbon mixtures originating from a steamcracking process. Further components present in such hydrocarbon mixtures are frequently also dimethylpentanes (DMP). In addition, these hydrocarbon mixtures may also already comprise the actual cyclohexane target product. In order, however, to obtain a pure target product, i.e. on-spec cyclohexane, the cyclohexane has to be separated from all other components still present in the hydrocarbon mixture used after the hydrogenation or isomerization, thus including the DMP present in the starting mixture. However, the separation of the DMP from cyclohexane, the actual process product, is technically quite demanding, and complex, especially where the 2,4-dimethylpentane (2,4-DMP) isomer of DMP is concerned. The standard boiling point of 2,4-DMP at 80.52° C. is very similar to the standard boiling point of cyclohexane (80.78° C.), whereas the standard boiling points of the other DMP isomers have a greater separation from cyclohexane (2,3-DMP has, for example, a standard boiling point of 89.88° C.).

U.S. Pat. No. 2,846,485 discloses a process for preparing high-purity cyclohexane and benzene, using a mixture comprising n-hexane, benzene, MCP, cyclohexane and DMP. In a first extractive distillation zone, benzene is separated from the other reactant components. The reactant which has been substantially freed of benzene is combined with a mixture which comprises cyclohexane and MCP and originates from the bottom of a second fractionating distillation zone. The mixture thus combined is fed into a first fractionating distillation zone, with removal of an MCP-containing fraction via the top and a cyclohexane-containing fraction from the bottom.

The overhead product of the first fractionating distillation zone is first conducted into an isomerization zone in which the majority of MCP is isomerized to cyclohexane using Friedel-Crafts catalysts such as aluminum chloride which may additionally comprise HCl. The isomerization product is introduced into the above-described second fractionating distillation zone, in order to remove n-hexane and low boilers as the top product therein. The bottom product from the first fractionating distillation zone is transferred into a second extractive distillation zone in which a cyclohexane-comprising mixture from the bottom is separated from the DMP drawn off via the top.

The process described in U.S. Pat. No. 2,846,485 is disadvantageous, since it is very complex in terms of apparatus (among other aspects). Cyclohexane, the actual process product, is not separated from DMP until the end of the process, since the cyclohexane formed in the isomerization of MCP has to be recycled into a DMP-containing fraction. In this process, moreover, the benzene is first removed in order to obtain it as an independent product. However, the removal of benzene is more complex in apparatus terms than the hydrogenation to give cyclohexane, which is present in one embodiment of the present invention.

U.S. Pat. No. 3,311,667 relates to a process for removing benzene from a mixture which is subsequently fed into an isomerization of MCP to cyclohexane. The hydrogenation involves hydrogenating benzene in the presence of a suitable catalyst, for example a metal catalyst on kieselguhr, with hydrogen to cyclohexane. The isomerization of MCP to cyclohexane is performed in the presence of metal halides such as acid-enhanced aluminum halide. However, U.S. Pat. No. 3,311,667 does not state that the material used for hydrogenation or isomerization may also comprise DMP. Consequently, this document also does not contain any statements as to the point at which DMP is removed from cyclohexane, or that this removal is problematic.

EP-A 1 995 297 discloses a process and a corresponding apparatus for hydrogenation and decyclization of benzene and the isomerization of C₅-C₆ paraffins present in a mixture comprising at most 1% by weight of benzene. For hydrogenation of benzene, metallic catalysts can be used, suitable metals being the elements of the platinum group, tin or cobalt and molybdenum. For isomerization of the mixture obtained in the hydrogenation, which may comprise a residual amount of benzene, zeolites in particular are used as the catalyst. In the process described in EP-A 1 995 297, the parameters in the isomerization are adjusted such that opening of the cyclohexane rings obtained in the benzene hydrogenation to isoalkanes is achieved. The primary purpose of this process is thus not the preparation of cyclohexane but the preparation of alkanes having a high degree of branching. Furthermore, EP-A 1 995 297 also does not contain any statements that the reactant used may comprise DMP, or that the separation of cyclohexane and DMP is problematic.

Ionic liquids are suitable, inter alia, as catalysts for the isomerization of hydrocarbons. A corresponding use of an ionic liquid is described, for example, in WO 2011/069929, where a specific selection of ionic liquids is used in the presence of an olefin for isomerization of saturated hydrocarbons, more particularly for isomerization of methylcyclopentane (MCP) to cyclohexane. A similar process is described in WO 2011/069957, but the isomerization therein is not effected in the presence of an olefin, but with a copper(II) compound.

US-A 2005/0082201 discloses a process for preparing gasoline with a low benzene content, wherein, in a first process step, a hydrocarbon mixture comprising benzene, olefins and sulfur compounds such as thiophenes is first fed into a distillation column, from which the low-boiling compounds are removed via the top, a benzene-containing fraction via a side draw and the high boilers from the column bottom. In a second process stage, the fraction obtained from the side draw is hydrogenated in the presence of a hydrogenation catalyst, converting benzene to cyclohexane and the thiophenes to hydrogen sulfide. The cyclohexane-containing mixture obtained in the second process stage is suitable for preparation of gasoline having a low benzene content. No isolation of the cyclohexane present therein or isomerization of MCP to cyclohexane is disclosed in US-A 2005/0082201. The same also applies to any presence of DMP in the starting mixture.

WO 2010/027987 relates to a further process for reducing the concentration of benzene in a hydrocarbonaceous mixture. In a first separation stage, a benzene-containing fraction comprising benzene and other C₆ hydrocarbons is separated from a high boiler fraction comprising carbons having seven or more carbon atoms. The benzene-containing fraction is subsequently hydrogenated to obtain a hydrocarbon fraction having a reduced benzene content. The hydrogenation of benzene forms cyclohexane. WO 2010/027987 also does not contain any pointers that cyclohexane can be isolated from the mixture obtained in the hydrogenation; instead, this process product too is to be used for gasoline production. This document likewise does not disclose isomerization of MCP to cyclohexane or the presence of DMP in the hydrogen starting mixture.

The performance of an extractive distillation for separation of close-boiling substances has already been known for some time and is disclosed, for example, in U.S. Pat. No. 4,053,369, U.S. Pat. No. 4,955,468 or WO 02/22528. The primary purpose of U.S. Pat. No. 4,053,369 is the performance of the extractive distillation as such, detached from any specific separation problem. As an example from a number of very many examples, the aforementioned documents also disclose the separation of DMP and cyclohexane by means of extractive distillation. However, these documents do not disclose the preparation of cyclohexane from benzene and/or MCP by hydrogenation or isomerization.

It is an object of the present invention to provide a novel process for preparing cyclohexane from a hydrocarbon mixture. In addition, it is to be possible to recover any cyclohexane present in the hydrocarbon mixture.

The object is achieved by a process for preparing cyclohexane, comprising the following steps:
a) feeding a hydrocarbon mixture (HM1) comprising
   i) benzene and/or methylcyclopentane (MCP) and
   ii) dimethylpentanes (DMP)
      into a distillation apparatus (D1),
b) removing a stream (S1) comprising DMP from the bottom of the distillation apparatus (D1),
c) removing a stream (S2) comprising benzene and/or MCP from an outlet of the distillation apparatus (D1), the outlet being arranged above the bottom of (D1),
d) feeding stream (S2) into at least one apparatus (V) suitable for hydrogenation and/or isomerization,
   the cyclohexane being prepared in the apparatus (V) by hydrogenation of benzene and/or by isomerization of MCP, and the distillation apparatus (D1) being arranged upstream of the apparatus (V).

### A BRIEF DESCRIPTION OF THE FIGURES

FIG. 1 illustrates the process according to the invention in a preferred embodiment of steps a) to d).

FIG. 2 illustrates an embodiment of the present invention.

FIG. 3 shows comparative example 2 schematically.

### DETAILED DESCRIPTION OF THE INVENTION

By virtue of the process according to the invention, it is advantageously possible to prepare pure cyclohexane from benzene and/or MCP if cyclohexane is prepared using hydrocarbon mixtures (starting mixtures) comprising not only benzene and/or MCP but also DMP.

A further advantage of the process according to the invention is considered to be that it can be performed very flexibly. According to the composition of the hydrocarbon mixture used (HM1/starting mixture), after DMP has been removed completely or at least substantially from (HM1) (“prior DMP removal”), a hydrogenation and/or an isomerization can be performed. The performance of a hydrogenation is required only if the starting mixture comprises benzene and possibly cyclohexene. The same applies to the performance of an isomerization if the starting mixture comprises MCP. If both a hydrogenation and an isomerization is performed, the sequence is as desired; preference is given in accordance with the invention to performing first the hydrogenation and then the isomerization.

Owing to the prior removal of DMP before the actual cyclohexane preparation process, the exceptionally complex separation, especially distillation, of DMP out of the cyclohexane process product can be avoided, especially when the DMP is 2,4-dimethylpentane (2,4-DMP) and it is present in the starting mixture in a concentration of >100 ppm. This distinctly reduces the energy intensity and apparatus complexity in the preparation of pure or high-purity cyclohexane.

The process according to the invention advantageously allows complete or virtually complete removal of the DMP present in the starting mixture by virtue of the prior removal from the starting mixture. Particular preference is given to performing the process according to the invention in such a way that the DMP present in the starting mixture is removed completely or virtually completely (down to 2% based on the amount of all DMP isomers present in the starting mixture) from the starting mixture by prior DMP removal. Alternatively, virtually complete DMP removal from the starting mixture can also be defined by the amount of DMP remaining in stream (S2) in relation to MCP and/or benzene. Taking this approach, it is especially preferable that the amount of DMP drawn off via the top in the distillation apparatus (D1) from stream (S2), based on the sum of the amounts of MCP and benzene drawn off via the top, is at most 0.1% by weight, preferably at most 0.02% by weight.

The process according to the invention can be performed irrespective of whether or not cyclohexane is already present in the hydrocarbon mixture (starting mixture) used. If cyclohexane itself is also present alongside DMP in the hydrocarbon mixtures used, this cyclohexane present in the starting mixture, in the process according to the invention, is removed via the bottom together with DMP. The disadvantage of a reduction in the amount of cyclohexane product, which is associated with this arrangement, however, is more than compensated for by the above-described reduction in energy intensity and apparatus complexity.

In one embodiment of the present invention, however, this cyclohexane present in the hydrocarbon starting mixture can be recovered. In this embodiment, the cyclohexane discharged from the process together with the DMP is removed again from DMP by distillation, preferably by an extractive or azeotropic distillation. The cyclohexane obtained, which is essentially free of DMP, can be fed back to the actual process product (cyclohexane which is prepared by the process according to the invention) or fed into the process according to the invention at another point. The advantage in the case of this process variant over a removal from a point further on in the process (downstream), i.e., for example, from the cyclohexane product stream, is considered to be that the DMP removal has to be conducted from a much smaller amount of cyclohexane, since DMP is removed only from the cyclohexane present in the hydrocarbon starting mixture and not also from the cyclohexane formed in the hydrogenation and/or isomerization, which is the actual process product. Accordingly, for this separate DMP/cyclohexane separation, smaller apparatuses and a smaller amount of energy are required.

In the context of the present invention, a distillation can be performed in all embodiments known to those skilled in the art (see, for example, Kirk-Othmer Encyclopedia of Chemical Technology Published Online: 2 Aug. 2004, Vol. 8 p. 786 ff.), for example also as an extractive distillation, azeotropic distillation or rectification. The respective distillation techniques are performed in the corresponding apparatuses known to those skilled in the art. For example, an extractive distillation generally comprises at least one extractive distillation column and at least one regeneration apparatus, which preferably likewise takes the form of a column and is connected downstream of the extractive distillation column. As already stated above, the performance of an extractive distillation for separation of close-boiling substances is described, for example, in U.S. Pat. No. 4,053,369, U.S. Pat. No. 4,955,468 or WO 02/22528.

In the context of the present invention, the term “rectification”, which is performed in a corresponding rectifying column (rectifying apparatus), also called rectification column or rectification apparatus, is understood to mean the following: in rectification, the vapor produced by distillation is conducted in countercurrent to a portion of the condensate thereof in a rectifying column. In this way, more volatile components are enriched in the top product and less volatile components in the bottom product of the rectifying column.

In the context of the present invention, the term “dimethylpentanes” (DMP) is understood to mean all known isomers of dimethylpentane, especially 2,2-dimethylpentane (2,2-DMP; standard boiling point: 79.17° C.), 2,3-dimethylpentane (2,3-DMP; standard boiling point: 89.88° C.), 3,3-dimethylpentane (3,3-DMP; standard boiling point: 86.09° C.) and 2,4-dimethylpentane (2,4-DMP; standard boiling point: 80.52° C.). This means that at least one dimethylpentane isomer is present in the corresponding mixtures or streams in the process according to the invention, preference being given to mixtures of two or more dimethylpentane isomers, one of these isomers preferably being 2,4-dimethylpentane.

In the context of the present invention, the term “compounds having a standard boiling point of 79 to 84° C.” is understood to mean all hydrocarbons which, at standard pressure, boil within the range from 79 to 84° C. and which, individually or as a mixture, may at first be present in the hydrocarbon mixture (HM1) in the process according to the invention. In the process according to the invention, one single compound or several of these compounds may be separated from one another. One single compound or several of these compounds may also be referred to separately in the text which follows as a constituent of mixtures or streams. If this is the case, only the specific compounds listed in each case are an obligatory constituent of the corresponding mixture or stream; the other compounds having a standard boiling point of 79 to 84° C. which are not named in the corresponding stream or mixture may (unless stated otherwise or no longer possible, for example owing to a preceding removal) likewise be present in the corresponding stream or mixture. One single compound or several of these compounds may also be covered by the definition of another selection of compounds, for example by the definition of the term “C₅-C₆-alkanes”.

Examples of compounds having a standard boiling point of 79 to 84° C. are cyclohexane (80.78° C.), 2,2-DMP (79.17° C.), 2,4-DMP (80.52° C.), 2,2,3-trimethylbutane (80.87° C.) and benzene (80.08° C.).

The same as stated above for the compounds having a standard boiling point of 79 to 84° C. also applies in the context of the present invention to compounds covered by the term “high boilers having a standard boiling point >84“C”. Examples of high boilers having a standard boiling point >84° C. are 3,3-DMP (86.09° C.), 2,3-DMP (89.88° C.), and the isoheptanes 2-methylhexane (2-MH; 90.06° C.), 3-methylhexane (3-MH; 91.87° C.) and 3-ethylpentane (3-EP; 93.45° C.).

In the context of the present invention, the two aforementioned groups of compounds (compounds having a standard boiling point of 79 to 84° C. and high boilers having a standard boiling point >84° C.) may also be combined to form one group of compounds. In this situation, the compounds are referred to correspondingly as “high boilers having a standard boiling point >78“C”. The above remarks regarding the two individual groups also apply analogously to this group of compounds.

The process according to the invention for preparing cyclohexane by hydrogenation of benzene and/or isomerization of MCP, with performance of a prior removal of DMP, is defined in detail hereinafter.

In the context of the present invention, in step a), a hydrocarbon mixture (HM1) comprising

i) benzene and/or methylcyclopentane (MCP) and
ii) dimethylpentanes (DMP)
is fed into a distillation apparatus (D1).

The individual components of the hydrocarbon mixture (HM1) may be present in any desired concentrations/ratios relative to one another. The hydrocarbon mixture (HM1) preferably comprises hydrocarbons having 5 to 8 carbon atoms in a proportion of at least 90% by weight, preferably at least 95% by weight. The hydrocarbons may be saturated or unsaturated and/or cyclic, linear or branched. More particularly, the hydrocarbon mixture (HM1) comprises between 10% by weight and 60% by weight, more preferably between 20% by weight and 50% by weight, of MCP and/or between 1% by weight and 30% by weight, more preferably between 4% by weight and 20% by weight, of benzene.

In a preferred embodiment of the present invention, the hydrocarbon mixture (HM1) additionally comprises cyclohexane. (HM1) preferably comprises
i) benzene,
ii) MCP,
iii) DMP,
iv) cyclohexane and
v) optionally at least one further compound selected from olefins and C₅-C₈-alkanes.

In component v) of the hydrocarbon mixture (HM1), the term “olefin” comprises, as well as linear, monounsaturated olefins such as pentene or hexene, also cyclic olefins, especially cyclohexene, and also dienes and cyclic dienes. In addition, the group of the C₅-C₈-alkanes also includes high boilers having a standard boiling point >84° C. or compounds having a standard boiling point of 79 to 84° C. The hydrocarbon mixture (HM1) may optionally also comprise hydrocarbons having more than eight carbon atoms and/or hydrocarbons having a relatively low boiling point, for example those having fewer than five carbon atoms.

Preferably, in the distillation apparatus (D1), the DMP present in the hydrocarbon mixture (HM1) is removed completely or virtually completely (down to 2% based on the amount of DMP present in (HM1)) from (HM1), especially from benzene and/or MCP (i.e. the main components of stream (S2)). Alternatively, virtually complete DMP removal from the starting mixture can also be defined by the amount of DMP remaining in stream (S2) in relation to MCP and/or benzene. Taking this approach, it is especially preferable that the amount of DMP drawn off via the top in the distillation apparatus (D1) from stream (S2), based on the sum of the amounts of MCP and benzene drawn off via the top, is at most 0.1% by weight, preferably at most 0.02% by weight.

The distillation apparatus (D1) is preferably a rectification column. It is additionally preferable that the outlet of the distillation apparatus (D1) from which stream (S2) is removed in step c) is above the feed with which the hydrocarbon mixture (HM1) is fed into (D1), the outlet preferably being in the top of (D1).

It is additionally preferable that the distillation apparatus (D1) does not comprise any reaction zone in which benzene and/or MCP are hydrogenated, and/or stream (S2) comprises at most 2%, preferably at most 0.5%, of cyclohexane (based on the amount present in the hydrocarbon mixture (HM1)).

In step b) of the process according to the invention, a stream (S1) comprising DMP is removed, preferably from the bottom of the distillation apparatus (D1). The specific composition of stream (S1) depends on the specific composition of the hydrocarbon mixture (HM1) used. Stream (S1) always necessarily comprises DMP. If the hydrocarbon mixture (HM1) used also comprises cyclohexane, stream (S1) generally comprises the majority of the cyclohexane from (HM1), preferably >90%.

The stream (S1) removed from the bottom of the distillation apparatus (D1) comprises DMP and possibly further components. The further components are preferably cyclohexane, high boilers having a standard boiling point >78° C. and/or unsaturated compounds. Some of the unsaturated compounds can also be regarded as high boilers having a standard boiling point >78° C. The unsaturated compounds are preferably selected from benzene, olefins, cyclic olefins, especially cyclohexene, dienes and cyclic dienes.

Stream (S1) preferably comprises at least 98% of the DMP present in the hydrocarbon mixture (HM1), more preferably at least 99% of the DMP. It is additionally preferable that the stream (S1) removed from the bottom of the distillation apparatus (D1) comprises at most 10%, preferably at most 5%, more preferably at most 2%, of the MCP present in (HM1).

In step c) of the process according to the invention, a stream (S2) comprising benzene and/or MCP is removed from an outlet of the distillation apparatus (D1), the outlet being arranged above the bottom of (D1). The specific composition of stream (S2) depends on the specific composition of the hydrocarbon mixture (HM1) used. Stream (S2) always necessarily comprises benzene or MCP, preferably benzene and MCP. If the hydrocarbon mixture (HM1) used also comprises cyclohexane, stream (S2) generally comprises only a portion of the cyclohexane from (HM1), preferably <10%.

Step c) is preferably performed in such a way that stream (S2) comprises at least 95%, preferably at least 98%, of the portion consisting of benzene and MCP present in the hydrocarbon mixture (HM1), and/or that stream (S2) comprises at most 0.1% by weight, preferably at most 0.02% by weight (based on the total amount of benzene and MCP in stream (S2)), of DMP. Stream (S2) more preferably comprises at most 0.015% by weight (based on the total amount of benzene and MCP in stream (S2)) of 2,4-DMP.

In step d) of the process according to the invention, stream (S2) is fed into at least one apparatus (V) suitable for hydrogenation and/or isomerization. In the apparatus (V), the cyclohexane is prepared by hydrogenation of benzene and/or by isomerization of MCP. In addition, the distillation apparatus (D1) is arranged upstream of the apparatus (V), and so the apparatus (V) is connected downstream of the distillation apparatus (D1).

According to the composition of the hydrocarbon mixture (HM1) used in step a) and consequently also of the stream (S2) which is fed into at least one apparatus (V), a hydrogenation and/or an isomerization is performed in accordance with the invention in step d). The performance of a hydrogenation is required only if (HM1) comprises benzene and possibly cyclohexene. The performance of an isomerization is in turn only required if (HM1) comprises MCP. If (HM1), in contrast, comprises both benzene (and possibly cyclohexene) and MCP, both a hydrogenation and an isomerization are performed in accordance with the invention. The hydrogenation and the isomerization are preferably performed spatially separated from one another, each in at least one separate apparatus (V) (see also the details further down in the text). The performance of a hydrogenation of benzene as such for preparation of cyclohexane and/or the performance of an isomerization of MCP as such for preparation of cyclohexane are known in principle to those skilled in the art.

In a first embodiment of the present invention, the apparatus (V) is at least one hydrogenation reactor (HR). In this embodiment, benzene is hydrogenated to cyclohexane in the hydrogenation reactor (HR), the hydrogenation preferably being effected using hydrogen. It is additionally preferable that the hydrogenation is effected in the liquid phase and/or in the presence of a nickel catalyst. This first embodiment is illustrated further in the text which follows.

The hydrogenation of benzene to cyclohexane is generally performed in the presence of a suitable catalyst. Suitable catalysts are in principle all catalysts known to those skilled in the art for this purpose, for example a metal catalyst on kieselguhr according to U.S. Pat. No. 3,311,667 or metallic catalysts according to EP A 1 995 297, wherein the metals used with preference are the elements of the platinum group, tin or cobalt and molybdenum.

Preference is given to performing the hydrogenation in the presence of a catalyst comprising, as an active metal (also referred to as metal component or active component), at least one element of groups 8 to 10 of the Periodic Table of the Elements (PTE), for example iron, cobalt, nickel or ruthenium (corresponds to transition group VIIIB of the CAS Version of the PTE), especially nickel or ruthenium. It is additionally preferable that the active metal is applied to a support material (support). Suitable supports are in principle all supports known to those skilled in the art, for example SiO₂-containing, zirconia-containing or alumina-containing supports. Particular preference is given to using a catalyst comprising nickel as an active metal on an alumina-containing support.

The hydrogenation as such is executed and operated in a manner known per se to those skilled in the art, preference being given to a combination of a main reactor operated in an optionally cooled circuit (recycling of a portion of the mixture flowing out of the reactor into the mixture flowing into the reactor, with optional positioning of the cooling unit upstream or downstream of said feed) and a downstream postreactor operated in straight pass, i.e. without recycling. In this case, the apparatus (V) thus comprises two hydrogenation reactors (HR).

The hydrogenation reactors (HR) are preferably designed as fixed bed reactors without internal cooling. In this case, the hydrogenation is preferably operated such that the temperature differential between entering and exiting mixture is monitored continuously and, when this value falls below a particular target value, the entrance temperature is raised. It is additionally preferable that the hydrogenation reactors are operated in trickle mode.

It is additionally preferable that the hydrogenation is followed downstream by an apparatus in which decompression is effected to a pressure below the pressure established in the postreactor. This affords a gas stream which comprises hydrogen dissolved beforehand in the hydrocarbon mixture and is in any case compressed and recycled into at least one of the hydrogenation reactors (HR).

The hydrogenation is preferably performed at a temperature between 50 and 200° C., more preferably between 100 and 180° C., and/or a pressure between 10 and 300 bar abs., more preferably between 30 and 200 bar abs.

It is additionally preferable in the process according to the invention that the overall conversion in the hydrogenation of the benzene (and of any other unsaturated compounds present in stream (S2)) is at least 90%, more preferably 99%, and/or the residual content of the benzene (and of any other unsaturated compounds present in stream (S2)) is 1% by weight, preferably at most 0.1% by weight, more preferably at most 0.01% by weight.

In a second embodiment of the present invention, the apparatus (V) is at least one apparatus (VAI) suitable for performance of an alkane isomerization. In this embodiment, MCP is isomerized to cyclohexane in the apparatus (VAI) suitable for performance of an alkane isomerization, preferably in the presence of an acidic ionic liquid. This second embodiment is illustrated further in the text which follows.

The isomerization of MCP to cyclohexane is generally performed in the presence of a suitable catalyst. Suitable catalysts are in principle all catalysts known for this purpose to those skilled in the art, for example Friedel-Crafts catalysts according to U.S. Pat. No. 2,846,485 such as aluminum chloride which may additionally contain HCl, or metal halides according to U.S. Pat. No. 3,311,667 such as aluminum chloride, zirconium chloride or boron trifluoride. Additionally suitable as catalysts are also the zeolites used in EP-A 1 995 297, or ionic liquids as used, for example, in WO 2011/069929.

In the context of the present invention, the isomerization is preferably effected in the presence of an acidic ionic liquid having the composition K1AlₙX₍₃ₙ₊₁₎ where K1 is a monovalent cation, X is halogen and 1<n<2.5. For example, mixtures of two or more acidic ionic liquids may be used, preference being given to using one acidic ionic liquid.

K1 is preferably an unsubstituted or at least partly alkylated ammonium ion or a heterocyclic (monovalent) cation, especially a pyridinium ion, an imidazolium ion, a pyridazinium ion, a pyrazolium ion, an imidazolinium ion, a thiazolium ion, a triazolium ion, a pyrrolidinium ion, an imidazolidinium ion or a phosphonium ion. X is preferably chlorine or bromine.

The acidic ionic liquid more preferably comprises, as a cation, an at least partly alkylated ammonium ion or a heterocyclic cation and/or, as an anion, a chloroaluminate ion having the composition AlₙCl₍₃ₙ₊₁₎ where 1<n<2.5. The at least partly alkylated ammonium ion preferably comprises one, two or three alkyl radicals (each) having one to ten carbon atoms. If two or three alkyl substituents are present with the corresponding ammonium ions, the respective chain length can be selected independently; preferably, all alkyl substituents have the same chain length. Particular preference is given to trialkylated ammonium ions having a chain length of one to three carbon atoms. The heterocyclic cation is preferably an imidazolium ion or a pyridinium ion.

The acidic ionic liquid especially preferably comprises, as a cation, an at least partly alkylated ammonium ion and, as an anion, a chloroaluminate ion having the composition AlₙCl₍₃ₙ₊₁₎ where 1<n<2.5. Examples of such particularly preferred acidic ionic liquids are trimethylammonium chloroaluminate and triethylammonium chloroaluminate.

Furthermore, in the isomerization, in addition to the acidic ionic liquid, it is also possible to use a hydrogen halide (HX) as a cocatalyst. The hydrogen halides (HX) used may in principle be any conceivable hydrogen halides, for example hydrogen fluoride (HF), hydrogen chloride (HCl), hydrogen bromide (HBr) or hydrogen iodide (HI). The hydrogen halides can optionally also be used as a mixture, but preference is given in the context of the present invention to using only one hydrogen halide. Preference is given to using the hydrogen halide whose halide moiety is also present in the above-described acidic ionic liquid (at least partly) in the corresponding anion. The hydrogen halide (HX) is preferably hydrogen chloride (HCl) or hydrogen bromide (HBr). The hydrogen halide (HX) is more preferably hydrogen chloride (HCl).

The apparatus (V) used for performance of the isomerization may in principle be any apparatuses known to the person skilled in the art for such a purpose. The apparatus (V) is preferably a stirred tank or a stirred tank cascade. A “stirred tank cascade” means that two or more, for example three or four, stirred tanks are connected in succession (in series).

The isomerization is preferably performed at a temperature between 0° C. and 100° C., especially preferably at a temperature between 30° C. and 60° C. It is additionally preferable that the pressure in the isomerization is between 1 and 20 bar abs. (absolute), preferably between 2 and 10 bar abs.

The performance of an isomerization of MCP in the presence of an acidic ionic liquid as a catalyst and optionally a hydrogen halide as a cocatalyst is known to those skilled in the art. The hydrocarbons (i.e. cyclohexane, MCP and any other hydrocarbons present in stream (S2)) and the ionic liquid in the isomerization preferably each form a separate phase, though portions of the ionic liquid may be present in the hydrocarbon phase and portions of the hydrocarbons in the ionic liquid phase. If present, the hydrogen halide, especially hydrogen chloride, is introduced, preferably in gaseous form, into the apparatus (V) for performance of the isomerization. The hydrogen halide may be present, at least in portions, in the two aforementioned liquid phases; the hydrogen halide preferably forms a separate, gaseous phase.

The isomerization is preferably performed in the apparatus (V) in such a way that two liquid phases and one gas phase are present in a stirred tank or a stirred tank cascade. The first liquid phase comprises the acidic ionic liquid to an extent of at least 90% by weight and the second liquid phase comprises the hydrocarbons to an extent of at least 90% by weight. The gas phase comprises at least one hydrogen halide, preferably hydrogen chloride, to an extent of at least 90% by weight. Optionally, a solid phase may also be present, this comprising components from which the ionic liquid is formed in solid form, for example AlCl₃. The pressure and composition of the gas phase are set here such that the partial pressure of the gaseous hydrogen halide, especially of HCl gas, in the gas phase is between 1 and 20 bar abs., preferably between 2 and 10 bar abs.

Preference is given to performing the process according to the invention with hydrocarbon mixtures (HM1) comprising both benzene and MCP. In this case (third embodiment of the process according to the invention), cyclohexane is prepared in step d)—on completion of prior DMP removal—by performing a hydrogenation and an isomerization of the starting mixture which has been completely or at least substantially freed of DMP. This is thus a combination of the above-described first and second embodiments according to step d), and the above-described elucidations for the first and second embodiments apply mutatis mutandis to the present third embodiment.

Optionally, the hydrogenation and the isomerization can be performed together in a single apparatus (V), preference being given, in this third embodiment of the process according to the invention, to performing the hydrogenation and the isomerization with spatial separation. The apparatus (V) preferably comprises at least one hydrogenation reactor (HR) (for the hydrogenation) and at least one apparatus (VAI) suitable for performance of an alkane isomerization. The sequence and number of stages are arbitrary, preference being given to performing first a hydrogenation and then an isomerization. The hydrogenation can be performed, for example, in one or two reactors connected in series (“two-stage”). The same applies to the isomerization, which can be performed, for example, in a stirred tank cascade of three or more stirred tanks connected in series.

In the process according to the invention, the cyclohexane obtained in the hydrogenation and/or isomerization in at least one apparatus (V) can be isolated. In general, cyclohexane is isolated from the apparatus (V) in a purity of at least 98% by weight, preferably at least 99.5% by weight, more preferably 99.9% by weight. If a plurality of apparatuses (V) are used, the cyclohexane is preferably isolated from the mixture obtained in the last apparatus (V), i.e. the apparatus (V) furthest downstream. The isolation itself is effected by methods known to those skilled in the art, for example using one or more distillation columns into which the cyclohexane-containing mixture present after the hydrogenation and/or isomerization in the apparatus (V) is fed. Optionally, the apparatus (V) configured according to the first, second, third or fourth (below) embodiment comprises, apart from the reaction apparatuses mentioned, also further devices, especially devices for separation, for example by means of rectification or distillation. Such devices (apparatuses) can be used, for example, to isolate cyclohexane and/or to remove high boilers from a mixture or the target product.

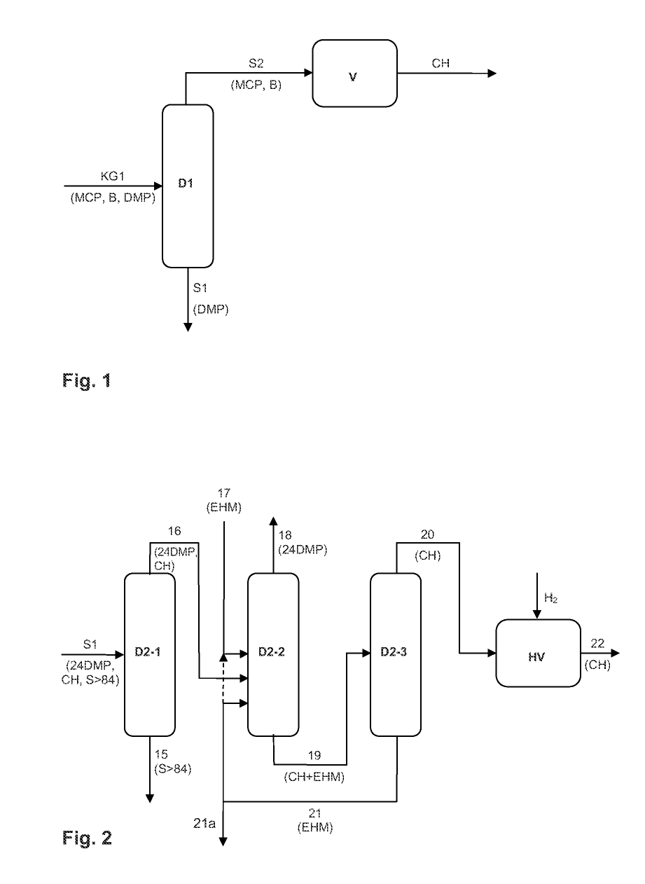

FIG. 1 once again illustrates the process according to the invention in a preferred embodiment of steps a) to d). CH means cyclohexane, B means benzene, and the bracketed expressions indicate the components most relevant to the process and/or the main components of the respective stream. Benzene and/or MCP may be present in the starting mixture (HM1) and correspondingly in stream (S2); benzene and MCP preferably are present. The apparatus (V) is, as described above, selected according to the composition of (HM1)/of stream (S2) (first to third embodiments). If both benzene and MCP are present, the apparatus (V) comprises both a hydrogenation and an isomerization (third embodiment). The hydrogenation is preferably performed in at least one reactor, and the isomerization preferably in a stirred tank or a stirred tank cascade, the isomerization being connected downstream of the hydrogenation.

In a further preferred embodiment (fourth embodiment) of the process according to the invention, any cyclohexane present in stream (S1) is separated from DMP, especially by distillation in a distillation apparatus (D2). This involves introducing stream (S1) into the distillation apparatus (D2), with separation of cyclohexane from DMP in (D2). The specific composition of stream (S1) has already been described above in connection with step b) of the invention.

The distillation or the distillation apparatus (D2) may have one or more stages, for example two or three stages; it preferably has three stages. In this context, the number of stages is understood to mean the number of columns, in each case including secondary apparatuses, for example reboilers and condensers, which together form the distillation apparatus (D2). A three-stage distillation apparatus (D2) thus means that a total of three columns, in each case including secondary apparatuses, for example reboilers and condensers, in each of which a distillation process can be performed, together form the distillation apparatus (D2). (D2) preferably comprises one extractive distillation column.

If the distillation apparatus (D2) comprises an extractive distillation column, the extractive distillation is preferably effected using an extraction aid (extraction assistant). The extraction aids used are generally compounds for which the following formula (I) applies:

γ_{DMP,E}^{∞}/γ_{CH,E}^{∞}>n (1)

where
γ_{DMP,E}^{∞}=activity coefficient of 2,4-dimethylpentane in the extraction aid at infinite dilution,
γ_{CH,E}^{∞}=activity coefficient of cyclohexane in the extraction aid at infinite dilution,
n=preferably 1.1, more preferably 1.3.

The extraction aids used are preferably oxygen-containing open-chain or cyclic organic compounds having a boiling point at least 5 K above that of cyclohexane (81° C.), especially those comprising an amide function R—CO—NR′R″ as a structural element, where R, R′ and R″ are (each independently) preferably selected from C₁-C₃₀-alkyl and H. Particularly suitable extraction aids are N-methylpyrrolidone and N-formylmorpholine. However, other compounds are also suitable, such as sulfolane, dimethyl sulfoxide and other compounds known to those skilled in the art as aprotic polar solvents. Also suitable are mixtures of a plurality of the compounds mentioned with one another or with water.

The cyclohexane/DMP separation preferably comprises the following steps i) to iii) and optionally step iv), the distillation apparatus (D2) being formed by the three components (D2-1) to (D2-3):
i) a rectifying column (D2-1) in which the majority of the high boilers having a standard boiling point >84° C. (based on the amount in the feed to (D2-1)) is removed via the bottom and the majority of the cyclohexane and other compounds having a standard boiling point of 79 to 84° C. (based on the amount in the feed to (D2-1)) via the top,
ii) an extractive distillation column (D2-2) in which the top product from (D2-1) is combined with an extraction aid and distilled in such a way that the majority of the extraction aid and of the cyclohexane is drawn off via the bottom and the majority of the other compounds having a standard boiling point of 79 to 84° C. present in the top product from (D2-1) is drawn off from (D2-2) via the top,
iii) a regeneration column (D2-3) in which the majority of the cyclohexane present in the bottom stream from (D2-2) is drawn off via the top and the majority of the extraction aid present in the bottom stream from (D2-2) via the bottom, and
iv) optionally a hydrogenation apparatus into which the top product from (D2-3) is conducted.

In the above embodiment, the term “majority” (unless stated otherwise) means at least 50% by weight, preferably at least 80% by weight, more preferably at least 95% by weight, especially at least 99% by weight (the values should be understood as proportions of the respective feed stream).

The optional step iv) included in the above embodiment is generally performed only when stream (S1) comprises unsaturated compounds which are thus also fed into the distillation apparatus (D2) and which are additionally not discharged from the process via the bottom of the rectifying column (D2-1). The hydrogenation in the optional step iv) can be performed analogously to the hydrogenation in the above-described first embodiment, preferably in one stage.

The above-described preferred embodiment of the cyclohexane/DMP separation can optionally also be performed without a rectifying column (D2-1) as an obligatory constituent. In this variant, the cyclohexane/DMP separation is effected analogously using only the two columns (D2-2) and (D2-3), in which case there may optionally also be a downstream hydrogenation apparatus. This variant is preferably performed if stream (S1) comprises only a small proportion of, if any, high boilers having a standard boiling point >84° C. If these high boilers are present, they are for the most part drawn off via the top of (D2-2) together with the other compounds having a standard boiling point of 79 to 84° C.

The above-described preferred embodiment using the extractive distillation column (D2-2) is preferably executed and operated in such a way that the DMP-containing stream drawn off via the top of (D2-2) comprises less than 50% by weight, preferably less than 10% by weight, of cyclohexane. In addition, the cyclohexane-containing stream drawn off via the top of regeneration column (D2-3) comprises preferably less than 1% by weight, more preferably less than 10 ppm by weight, of extraction aid and/or less than 1% by weight, preferably less than 300 ppm by weight, of dimethylpentanes, more preferably less than 150 ppm by weight of 2,4-dimethylpentane.

It is additionally preferable that cyclohexane is isolated from (D2) in a purity of at least 98% by weight, especially at least 99.5% by weight. With regard to the performance of the isolation of the cyclohexane, the same considerations apply as detailed above in connection with the isolation of the cyclohexane from the apparatus (V). Alternatively, the cyclohexane originating from the distillation apparatus (D2) in the fourth embodiment can be combined with the cyclohexane which has been prepared in the apparatus (V) in the first to third embodiments.

The above-described fourth embodiment of the present invention is additionally illustrated hereinafter in a preferred embodiment in conjunction with FIG. 2. In FIG. 2, the abbreviations, arrows and other symbols have analogous meanings to those explained above for FIG. 1 or in the description of this preferred embodiment. In the embodiment according to FIG. 2, a distillation apparatus (D2) consisting essentially of three columns ((D2-1) to (D2-3)) with a hydrogenation apparatus connected downstream is used. The individual columns may additionally also have secondary apparatuses, such as reboilers or condensers, which are not shown in FIG. 2 for the sake of clarity. HV means hydrogenation apparatus, EHM means extraction aid, S>84 means high boilers having a standard boiling point >84° C., 24DMP means 2,4-dimethylpentane and the bracketed expressions indicate the components most relevant to the process and/or the main components of the respective stream. 24DMP is mentioned by way of example as a preferred component of the (other) compounds having a standard boiling point of 79 to 84° C. The extraction aid used is preferably N-methyl-2-pyrrolidone (NMP).

Stream (S1) which originates with preference from the bottom of the distillation apparatus (D1) and comprises DMP, cyclohexane, unsaturated compounds and possibly high boilers having a standard boiling point >78° C. is fed into the rectifying column (D2-1). The unsaturated compounds are preferably selected from benzene, olefins, cyclic olefins, especially cyclohexene, dienes and cyclic dienes. In (D2-1), the cyclohexane present in stream (S1) is concentrated, by first separating stream (S1) by means of rectification into a stream 15 enriched in higher-boiling components than cyclohexane (i.e., for example, 3,3-DMP and other high boilers having a standard boiling point >84° C. or unsaturated compounds having corresponding boiling points) and a stream 16 depleted of higher-boiling components than cyclohexane (stream 16 thus comprises cyclohexane and a majority of the other compounds having a boiling point of 79 to 84° C., at least a portion of the unsaturated compounds and a residual amount of high boilers having a standard boiling point >84° C.). Stream 15 can, for example, be conducted as a cofeed to a steamcracking process or be used as a constituent of fuel mixtures.

Stream 16 is conducted into an extractive distillation column (D2-2). At a point above the feed of stream 16, a stream 17 comprising at least one extraction aid (EHM) is conducted into the extractive distillation column (D2-2). At a point likewise above the feed of stream 16, preferably above the feed of stream 17, for example at the top of the column or downstream of the top condenser of the column, a stream 18 enriched in other compounds having a standard boiling point of 79 to 84° C., especially in 2,4-DMP, compared to stream 16 is withdrawn. Stream 18 preferably comprises a majority of the other compounds having a standard boiling point of 79 to 84° C., especially of 2,4-DMP, present in stream 16. Via a point below the feed of stream 16, preferably via the column bottom, a stream 19 comprising the extraction aid, cyclohexane and the unsaturated compounds is withdrawn, the concentration ratio of cyclohexane/other compounds having a standard boiling point of 79 to 84° C., especially 2,4-DMP, being higher in stream 19 than in stream 16.

The extractive distillation column (D2-2) is preferably executed and operated in such a way that stream 18 comprises at most 100 ppm by weight, preferably at most 10 ppm by weight, more preferably at most 1 ppm by weight, of extraction aid. This can be achieved by virtue of the highest feed of an EHM-containing stream being at least 5, preferably at least 10, theoretical plates (as per the definition known to those skilled in the art) below the top and/or (D2-2) being operated with a reflux ratio of at least 5, preferably at least 10.

Stream 19, optionally after preheating, is conducted into the regeneration column (D2-3). From the regeneration column (D2-3), a stream 20 enriched in cyclohexane compared to stream 19 and a stream 21 depleted of cyclohexane compared to stream 19 (stream 21 comprises primarily the extraction aid, a portion of cyclohexane and any residual amount of other compounds having a standard boiling point of 79 to 84° C., especially of 2,4-DMP) are drawn off. From stream 21, a discharge stream (purge stream) 21 a is branched off, this making up preferably not more than 5%, more preferably not more than 1%, of the amount of stream 21. The remaining stream, optionally after cooling (which can also be effected in a thermally integrated system with a preheating of stream 19), is supplied at least partly to stream 17 and/or recycled into the extractive distillation column (D2-2) in the vicinity of stream 16.

Stream 20 is optionally, together with a hydrogen-comprising stream, conducted into the hydrogenation apparatus (HV) in which, with the aid of a suitable catalyst, the unsaturated compounds selected from benzene, olefins, cyclic olefins, especially cyclohexene, dienes and cyclic dienes, are hydrogenated. Hydrogen can also be introduced into (HV) separately from stream 20, as shown in FIG. 2. The stream 22 obtained in the hydrogenation comprises cyclohexane as the main constituent and can optionally be worked up further; for example, on-spec (high-purity) cyclohexane can be isolated from stream 22. Stream 22 can optionally also be combined with the cyclohexane or a cyclohexane-containing stream which is prepared in the process according to the invention in apparatus (V) (in the first to third embodiments).

In the context of the present invention, preference is given to performing a combination of the above-described third and fourth embodiments.

The present invention is to be illustrated hereinafter by examples.

For the simulation calculation, BASF's own Chemasim software was used (in the case of use of the commercially available Aspen Plus software (manufacturer: Aspen Tech, Burlington, Mass., USA), the same results would be obtained). The following rough composition of the hydrocarbon mixture (KG1) is used (for detailed composition, see Table 1):

| | |
|---|---|
| Benzene | 9.6% by weight |
| Methylcyclopentane (MCP) | 32.0% by weight |
| Dimethylpentane (DMP) | 0.8% by weight |
| Other hydrocarbons | 57.6% by weight |

In the apparatus (V), in Examples 1 and 2 hereinafter, first a hydrogenation and then an isomerization are carried out. The relevant parameters for these reactions are as follows:
1. Hydrogenation: Ni fixed-bed catalyst
   36 bar H₂
   reaction temperature: 140-160° C.
2. Isomerization: hydrocarbon conversion in the presence of an ionic liquid (trimethylammonium heptachlorodialuminate)
   reaction pressure: 3.5 bara HCl
   reaction temperature: 50° C.

In the two examples, the distillation apparatus D1 is located in one case upstream of the apparatus V (Example 1) and in one case downstream of the apparatus V (Comparative Example 2).

1. Example with Upstream Removal of DMP

Example 1 is carried out in accordance with the embodiment shown schematically in FIG. 1.

The stated hydrocarbon mixture KG1 (for detailed composition, see Table 1) is introduced into a distillation apparatus D1 (rectifying column). In this column, the DMP contained in KG1 is removed via the liquid phase of D1 (S1) in such a way that the stream S2 subsequently contains not more than 0.1% by weight of DMP, based on benzene and MCP (see S2, Table 1). The stream S2 is taken off as distillate from D1 and contains the corresponding benzene and MCP. This stream is subsequently introduced into an apparatus V, in which cyclohexane is prepared by hydrogenation of benzene and by isomerization of MCP. The cyclohexane prepared leaves apparatus V with a purity of at least 99.9% by weight (see CH, Table 1).

TABLE 1
Properties and composition of the streams from Example 1

| | | | Stream: | | | |
|---|---|---|---|---|---|---|
| Topology | | unit | KG1 | S1 | S2 | CH |
| from apparatus | | | | D1 | D1 | V |
| to apparatus | | | D1 | | V | |
| Phase | | | flow | flow | flow | flow |
| Properties | | | inst. value | inst. value | inst. value | inst. value |
| Temperature | | ° C. | 25 | 87 | 64 | 40 |
| Pressure | | bar | 1 | 1 | 1 | 1 |
| Enthalpy | | kW | 67 | 54 | 140 | 43 |
| Mass flow rate | | kg/h | 5000 | 1064 | 3936 | 2129 |
| Concentrations | Mol. M. | | inst. value | inst. value | inst. value | inst. value |
| CYCLOPENTANE | 70.135 | g/g | 0.09493 | 0.00000 | 0.12059 | 0.00000 |
| 2-M-PENTANE | 86.179 | g/g | 0.06739 | 0.00000 | 0.08561 | 0.00000 |
| 3-M-PENTANE | 86.179 | g/g | 0.04333 | 0.00000 | 0.05503 | 0.00000 |
| HEXANE | 86.179 | g/g | 0.14966 | 0.00003 | 0.19009 | 0.00000 |
| M-CY-PENTANE | 84.163 | g/g | 0.32621 | 0.00868 | 0.41201 | 0.00013 |
| CYCLOHEXANE | 84.163 | g/g | 0.08849 | 0.41498 | 0.00027 | 0.99950 |
| HEPTANE | 100.21 | g/g | 0.02436 | 0.11450 | 0.00000 | 0.00000 |
| 2-M-HEXANE | 100.21 | g/g | 0.02875 | 0.13514 | 0.00000 | 0.00009 |
| C13DMCPENTANE | 98.19 | g/g | 0.00938 | 0.04409 | 0.00000 | 0.00000 |
| M-CYCLOHEXANE | 98.19 | g/g | 0.00689 | 0.03238 | 0.00000 | 0.00000 |
| BENZENE | 78.115 | g/g | 0.09628 | 0.01070 | 0.11940 | 0.00000 |
| 2,4DM-PENTANE | 100.21 | g/g | 0.00608 | 0.02836 | 0.00006 | 0.00002 |
| 3-M-HEXANE | 100.21 | g/g | 0.02582 | 0.12136 | 0.00000 | 0.00001 |
| 2,2DM-PENTANE | 100.206 | g/g | 0.00237 | 0.01066 | 0.00014 | 0.00025 |

Column D1

| | |
|---|---|
| Number of separation stages | 80 |
| Reflux ratio | 4.159 |
| Reboiler power [kW] | 2003 |
| Condenser power [kW] | 2005 |

To ensure the removal of the DMP in the column D1, 80 stages are required, with a reboiler power of approximately 2 MW.

2. Comparative Example with Downstream Removal of DMP

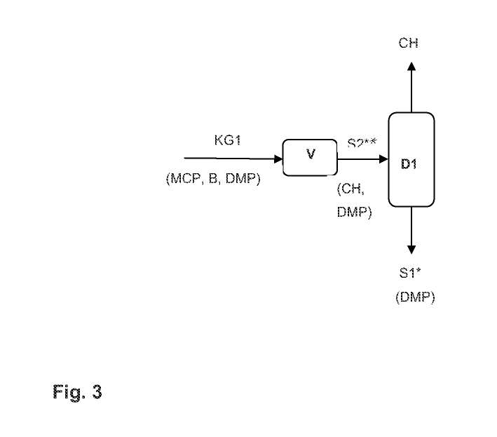

Comparative Example 2 is shown schematically in FIG. 3. Unless otherwise indicated, the abbreviations and symbols in FIG. 3 have the same definitions as described above for FIG. 1 or 2.

The stated hydrocarbon mixture KG1 (for detailed composition, see Table 1) is in this case first fed into the apparatus V, in which cyclohexane is prepared by hydrogenation of benzene and by isomerization of MCP. In this case the DMP is not removed in an upstream column. The stream S2*, coming from the hydrogenation and isomerization stage, is now introduced into a distillation apparatus (rectifying column). In this column, the DMP contained in S2* is removed via the liquid phase as stream S1*, thus giving a purity of 98% by weight for CH in the distillate. The detailed compositions of the individual streams are found in Table 2.

TABLE 2
Properties and composition of the streams from Comparative
Example 2

| | | | Stream: | | | |
|---|---|---|---|---|---|---|
| Topology | | unit | KG1 | S2* | S1* | CH |
| from apparatus | | | | V | D1 | D1 |
| to apparatus | | | V | D1 | | |
| Phase | | | flow | flow | flow | flow |
| Properties | | | inst. value | inst. value | inst. value | inst. value |
| Temperature | | ° C. | 25 | 40 | 93.12315 | 80.307801 |
| Pressure | | bar | 1 | 1.1 | 1 | 1 |
| Enthalpy | | kW | 67 | 66 | 33 | 111 |
| Mass flow rate | | kg/h | 5000 | 3178 | 580 | 2598 |
| Concentrations | Mol. M. | | inst. value | inst. value | inst. value | inst. value |
| CYCLOPENTANE | 70.135 | g/g | 0.09493 | 0.00000 | 0.00000 | 0.00000 |
| 2-M-PENTANE | 86.179 | g/g | 0.06739 | 0.00000 | 0.00000 | 0.00000 |
| 3-M-PENTANE | 86.179 | g/g | 0.04333 | 0.00000 | 0.00000 | 0.00000 |
| HEXANE | 86.179 | g/g | 0.14966 | 0.00000 | 0.00000 | 0.00000 |
| M-CY-PENTANE | 84.163 | g/g | 0.32621 | 0.00046 | 0.00000 | 0.00056 |
| CYCLOHEXANE | 84.163 | g/g | 0.08849 | 0.81202 | 0.00673 | 0.99178 |
| HEPTANE | 100.21 | g/g | 0.02436 | 0.03828 | 0.20977 | 0.00000 |
| 2-M-HEXANE | 100.21 | g/g | 0.02875 | 0.05125 | 0.28085 | 0.00000 |
| C13DMCPENTANE | 98.19 | g/g | 0.00938 | 0.00733 | 0.04017 | 0.00000 |
| M-CYCLOHEXANE | 98.19 | g/g | 0.00689 | 0.03048 | 0.16702 | 0.00000 |
| BENZENE | 78.115 | g/g | 0.09628 | 0.00000 | 0.00000 | 0.00000 |
| 2,4DM-PENTANE | 100.21 | g/g | 0.00608 | 0.00254 | 0.00000 | 0.00311 |
| 3-M-HEXANE | 100.21 | g/g | 0.02582 | 0.04143 | 0.22705 | 0.00000 |
| 2,2DM-PENTANE | 100.206 | g/g | 0.00237 | 0.00372 | 0.00000 | 0.00455 |

Column D1 - Example 2

| | |
|---|---|
| Number of separation stages | 200 |
| Reflux ratio | 50 |
| Reboiler power [kW] | 13082 |
| Condenser power [kW] | 13003 |

To ensure the purity of 98% by weight for CH, 200 stages are needed for column D1, with a reboiler power of approximately 13 MW.

### 3. Results of the Simulation Calculation

The results show that as a result of the upstream removal of the high boilers (DMP), it is possible to achieve a higher purity of cyclohexane for significantly lower investment (80 stages versus 200 stages) and variable costs (2 MW versus 13 MW).

1. A process for preparing cyclohexane, comprising the following steps:
a) feeding a hydrocarbon mixture (HM1) comprising
i) benzene and/or methylcyclopentane (MCP) and
ii) dimethylpentanes (DMP)
into a distillation apparatus (D1),
b) removing a stream (S1) comprising DMP from the bottom of the distillation apparatus (D1),
c) removing a stream (S2) comprising benzene and/or MCP from an outlet of the distillation apparatus (D1), the outlet being arranged above the bottom of (D1),
d) feeding stream (S2) into at least one apparatus (V) suitable for hydrogenation and/or isomerization,
the cyclohexane being prepared in the apparatus (V) by hydrogenation of benzene and/or by isomerization of MCP, and the distillation apparatus (D1) being arranged upstream of the apparatus (V).

2. The process according to claim 1, wherein cyclohexane is isolated from the apparatus (V) in a purity of at least 98% by weight, preferably of at least 99.5% by weight, more preferably of at least 99.9% by weight.

3. The process according to claim 1 or 2, wherein the hydrocarbon mixture (HM1) additionally comprises cyclohexane, (HM1) preferably comprising
i) benzene,
ii) MCP,
iii) DMP,
iv) cyclohexane and
v) optionally at least one further compound selected from olefins and C₅-C₈-alkanes.

4. The process according to any of claims 1 to 3, wherein stream (S2) comprises at least 95%, preferably at least 98%, of the portion consisting of benzene and MCP present in the hydrocarbon mixture (HM1), and/or stream (S2) comprises at most 0.1% by weight, preferably at most 0.02% by weight (based on the total amount of benzene and MCP in stream (S2)), of DMP, stream (S2) more preferably comprising at most 0.015% by weight (based on the total amount of benzene and MCP in stream (S2)) of 2,4-DMP.

5. The process according to any of claims 1 to 4, wherein the distillation apparatus (D1) is a rectification column, and the outlet of the distillation apparatus (D1) from which stream (S2) is removed in step c) is above the feed with which the hydrocarbon mixture (HM1) is fed into (D1), the outlet preferably being in the top of (D1).

6. The process according to any of claims 1 to 5, wherein the distillation apparatus (D1) does not comprise any reaction zone in which benzene and/or MCP are hydrogenated, and/or stream (S2) comprises at most 2%, preferably at most 0.5%, of cyclohexane (based on the amount present in the hydrocarbon mixture (HM1)).

7. The process according to any of claims 1 to 6, wherein the stream (S1) removed from the bottom of the distillation apparatus (D1) comprises DMP, preferably at least 98% of the DMP present in the hydrocarbon mixture (HM1), and/or the stream (S1) removed from the bottom of the distillation apparatus (D1) comprises at most 10%, preferably at most 5%, more preferably at most 2%, of the MCP present in (HM1).

8. The process according to any of claims 3 to 7, wherein stream (S1) is introduced into a distillation apparatus (D2), with separation of cyclohexane from DMP in (D2), (D2) preferably comprising one extractive distillation column and/or cyclohexane preferably being isolated from (D2) in a purity of 98% by weight, especially at least 99.5% by weight.

9. The process according to claim 8, wherein the cyclohexane/DMP separation comprises the following steps i) to iii) and optionally step iv), the distillation apparatus (D2) being formed by the three components (D2-1) to (D2-3):
i) a rectifying column (D2-1) in which a majority of the high boilers having a standard boiling point >84° C. (based on the amount in the feed to (D2-1)) is removed via the bottom and a majority of the cyclohexane and other compounds having a standard boiling point of 79 to 84° C. (based on the amount in the feed to (D2-1)) via the top,
ii) an extractive distillation column (D2-2) in which the top product from (D2-1) is combined with an extraction aid and distilled in such a way that the majority of the extraction aid and of the cyclohexane is drawn off via the bottom and the majority of the other compounds having a standard boiling point of 79 to 84° C. present in the top product from (D2-1) is drawn off from (D2-2) via the top,
iii) a regeneration column (D2-3) in which the majority of the cyclohexane present in the bottom stream from (D2-2) is drawn off via the top and the majority of the extraction aid present in the bottom stream from (D2-2) via the bottom, and
iv) optionally a hydrogenation apparatus into which the top product from (D2-3) is conducted.

10. The process according to claim 8 or 9, wherein cyclohexane originating from the distillation apparatus (D2) is combined with the cyclohexane which has been prepared in the apparatus (V).

11. The process according to any of claims 1 to 10, wherein the apparatus (V) is at least one hydrogenation reactor (HR) and/or the apparatus (V) is at least one apparatus (VAI) suitable for performance of an alkane isomerization.

12. The process according to claim 11, wherein benzene is hydrogenated to cyclohexane in the hydrogenation reactor (HR), preferably in the presence of a nickel catalyst.

13. The process according to claim 11 or 12, wherein MCP is isomerized to cyclohexane in the apparatus (VAI) suitable for performance of an alkane isomerization, preferably in the presence of an acidic ionic liquid.
